(19) United States
(12) Patent Application Publication
(10) Pub. No.: US 2009/0216073 A1
(43) Pub. Date: Aug. 27, 2009

(54) Devices and Methods for Placing Slings and Other Materials

(76) Inventor: Ralph Zipper, Melbourne, FL (US)

Correspondence Address: HAYWORTH, CHANEY & THOMAS, P.A., 202 N. HARBOR CITY BLVD., SUITE 300, MELBOURNE, FL, 32935, US

(21) Appl. No.: 12/393,702

(22) Filed: Feb. 26, 2009

Related U.S. Application Data
(60) Provisional application No. 61/067,306, filed on Feb. 27, 2008.

(51) Int. Cl. A61F 2/00

(52) U.S. Cl. 600/30

(57) ABSTRACT

New methods for inserting slings and other material, wherein such methods may comprise a needle creating a pathway from a first insertion point to a first exit point that may thereafter be utilized as a second insertion point allowing for a change in direction to the needle pathway. A plurality of additional exit and re-insertion points may be repeated to provide a longer circuitous pathway, if so desired. A malleable needle may further allow unique pathways and needle angles when the needle's shape may be adjustable at each respective insertion point. A tapered device may form a final exit pathway having a tapered and reduced diameter that provides a greater friction fit between the implanted material and surrounding tissue. The present inventive method may further incorporate sling ends terminating from previously unused regions of the body including but not limited to the perineum, preprepubic, and supraobturator regions.

### CROSS REFERENCE TO RELATED APPLICATIONS

This application claims the benefit of provisional patent application Ser. No. 61/067,306, filed with the USPTO on Feb. 27, 2008, which is herein incorporated by reference in its entirety.

### STATEMENT REGARDING FEDERALLY SPONSORED RESEARCH OR DEVELOPMENT

Not applicable.

### INCORPORATION-BY-REFERENCE OF MATERIAL SUBMITTED ON A COMPACT DISK

Not applicable.

### BACKGROUND OF THE INVENTION

1. Field of the Invention

The present invention generally relates to methods for the placement of materials within a patient, more specifically, the present invention relates to methods for placing material for the treatment of urinary incontinence, fecal incontinence, pelvic organ prolapse, and the like.

2. Background Art

Anatomical tissues may become weakened or damaged by age, injury, or disease. This decrease in the structural integrity of anatomical tissues may have significant medical consequences. Even in the absence of tissue necrosis, weakening of an anatomical structure may impair one or more of the biological functions of the tissue. To help alleviate this impact on biological function, implantable, supportive slings have been developed. These slings can be implanted into a patient to provide support for the weakened or damaged tissue. The support provided by the sling mimics the natural position and structure of the tissue, and thereby helps decrease or eliminate impairment of biological function resulting from tissue weakening or damage. Although supportive slings have been used in numerous contexts to address the weakening of a variety of anatomical tissues, they have proven particularly useful for decreasing urinary incontinence resulting from weakening or damage to urethral, periurethral, and/or bladder tissue.

Stress urinary incontinence (SUI) affects primarily women, but also men, and is generally caused by two conditions, intrinsic sphincter deficiency (ISD) and hypermobility. These conditions may occur independently or in combination. In ISD, the urinary sphincter valve located within the urethra fails to close properly causing urine to leak out of the urethra during stressful activity. Hypermobility is a condition in which the pelvic floor is distended, weakened, or damaged, causing the bladder neck and proximal urethra to rotate and descend in response to increases in intra-abdominal pressure (e.g., due to sneezing, coughing, straining, etc.). As a result, the patient's response time becomes insufficient to promote urethral closure and, consequently, the patient suffers from urine leakage and/or flow. SUI has a variety of causes including, but not limited to, pregnancy, aging, infection, injury, congenital defects, and disease.

A popular treatment of SUI involves placement of an implantable sling under the bladder neck or the mid-urethra to provide a urethral platform. Placement of the sling limits the endopelvis fascia drop. There are various methods for placing the sling. Slings can be affixed and stabilized using traditional bone anchoring approaches, as well as recently developed anchor-less methods. Additionally, a variety of implantation procedures, including various routes of administration, exist. These procedures provide physicians with a range of implantation options. Physicians can readily select amongst the various implantation procedures based on numerous patient-specific factors including, but not limited to, age, gender, overall health, location of tissue defect, the degree of tissue impairment, and the like. Furthermore, physicians can select from amongst numerous sling delivery devices that facilitate sling placement.

Slings differ in the type of implantable material and anchoring methods. In some cases, the sling is placed under the bladder neck or urethra and secured via suspension sutures to a point of attachment (e.g. bone or ligament) through an abdominal and/or vaginal incision. In other instances, no securing suture is used.

Complications associated with procedures for treating incontinence include urinary retention, bladder instability and erosion of an implanted article into surrounding tissue. See Spencer et al, A Comparison of Endoscopic Suspension of the Vesical Neck With Suprapubic Vesicourethropexy for Treatment of Stress Urinary Incontinence, J. Urol. 137: 411, (1987); Araki et al., The Loop Loosening Procedure for Urination Difficulties After Stamey Suspension of the Vesical Neck, J. Urol., 144; (1990); and Webster et al., Voiding Dysfunction Following Cystourethropexy: Its Evaluation and Management, J. Urol., 144; (1990).

With respect to sling procedures, if the sling mesh is too loosely associated with its intended physiological environment, the mesh may be ineffective in supporting the urethra and treating incontinence. Several complications can also arise from a mesh that is too tightly placed including retention, sling erosion and other damage to surrounding tissue such as the urethra and vagina.

Proper tension of a sling is an important factor for a successful surgical procedure. Surgical approaches to applying tension or slack in a sling procedure vary widely. See Decter, Use of the Fascial Sling for Neurogenic Incontinence: Lessons Learned, The Journal of Urology, Vol. 150, 683-686 (1993).

Despite the numerous advances in sling design, implantation methodologies, and delivery devices, no single method and/or device is appropriate for every situation. Accordingly, devices, systems, and methods that offer new approaches for sling implantation would be advantageous to the medical community. The present invention provides for methods of sling placement that further decreases the risks of injury and other complications and the present disclosure further provides instrumentation to facilitate the present inventive methods.

### BRIEF SUMMARY OF THE INVENTION

In accordance with one embodiment of the present invention, a method of placing material attached to a needle in a circuitous pathway through bodily tissue comprising the steps of inserting the needle into bodily tissue at a first insertion point, advancing the needle in a first trajectory through the bodily tissue, exiting the needle from the bodily tissue at a first exit point, inserting the needle into the bodily tissue at the first exit point, advancing the needle in a second trajectory through the bodily tissue, wherein the second trajectory is distinct from the first trajectory, and exiting the needle from the bodily tissue at a second exit point.

In accordance with another embodiment of the present invention, a method of placing material attached to a tapered needle body in a circuitous pathway through bodily tissue comprising the steps of inserting the tapered needle body into the bodily tissue at a first insertion point, advancing the tapered needle body in a first trajectory through the bodily tissue, creating a tapered needle pathway within the bodily tissue adjacent a first exit point, and advancing the material through the tapered needle pathway within the bodily tissue, wherein the tapered needle pathway constricts or restrains movement of the material disposed there through.

In accordance with still another embodiment of the present invention, a method of placing material attached to a needle in a circuitous pathway through bodily tissue comprising the steps of inserting the needle into the bodily tissue at a first insertion point, advancing the needle into the bodily tissue, exiting the needle from the bodily tissue at a first transient exit point, inserting the needle into the bodily tissue at a second insertion point, advancing the needle into the bodily tissue, and exiting the needle from the bodily tissue at a second exit point.

### BRIEF DESCRIPTION OF THE DRAWINGS

FIGS. 1A-1F depict perspective views of an embodiment of method steps of the present invention for placing material within a patient's body.

FIGS. 2A-2B depict perspective views of an embodiment of method steps of the present invention using a malleable needle for placing material within a patient's body.

FIGS. 3A-3B depict side cross sectional views an embodiment of a device of the present invention functioning to deliver or retrieve a material within a patient's body.

FIG. 4 depicts a perspective view of an end result after performing an embodiment of method steps of the present invention for placing material within a patient's body.

FIG. 5 depicts a side view of typical pathways for slings generally having a terminus in either the suprapubic space or retropubic space.

FIG. 6 depicts a side view of an end result after performing method steps of the present invention for placing material within a patient's body wherein the material has a terminus in the perineum space.

FIG. 7 depicts a side view of an end result after performing method steps of the present invention for placing material within a patient's body wherein the material has a terminus in the preprepubic space.

FIG. 8 depicts a perspective view of an end result after performing method steps of the present invention for placing material within a patient's body wherein the material has a terminus in the supraobturator space.

FIG. 9 depicts a perspective view of an end result after performing method steps of the present invention for placing material within a patient's body wherein the material has a first terminus in the supraobturator space and a second terminus in the preprepubic space.

FIG. 10 depicts a perspective view of an the end result of FIG. 9 further illustrating the dorsal nerve of the clitoris and crus as being disposed deep to the bone and safe from injury from the present inventive methods.

FIG. 11 depicts a perspective view of an end result after performing method steps of the present invention wherein the path of the inserted material does not penetrate the obturator foramen thus removing the risk of obturator vessel and/or nerve injury from the present inventive methods.

### DETAILED DESCRIPTION OF THE INVENTION

Stress urinary incontinence is broadly defined as the involuntary loss of urine with activities that cause an increase in intraabdominal pressure. Examples of such activities include coughing, laughing, sneezing, bending, yelling, exercising, and the like. Sling procedures are a broad class of surgical procedures, which are used to treat stress urinary incontinence. All sling procedures involve the placement of natural (e.g. patient's own tissue) or synthetic material around or near the urethra. This material is referred to as the sling. When the sling is placed appropriately and does not move following the procedure, the patient will be cured of stress urinary incontinence.

All methods of sling placement, also called sling procedures, have associated complications. The most common complications are injury to the bladder, bowel, blood vessels and/or nerves. Some recent sling procedures have attempted to decrease the incidence of complications by choosing sling pathways that avoid the more injury prone structures. However, injury to nerves, blood vessels, and other such structures still occur. When these injuries occur, they can produce rather serious complications.

Pelvic organ prolapse is a disorder relating to pelvic organ support. This may be defined as a herniation of the pelvic organs into or beyond the normal vaginal space. Examples of such disorders include but are not limited to cystoceles, rectoceles, uterine prolapse, and vaginal wall prolapse. Newer treatments of such disorders may utilize natural and/or synthetic materials to create support for the pelvic organs. Methods of placement of synthetic and/or natural material for the treatment of pelvic organ prolapse and the like have identical concerns and complication issues as those described above for sling procedures for the treatment of urinary incontinence.

The present inventive methods provide for the avoidance of the majority of vital structures disposed in the body region of the procedure. Such vital structures may include but are not limited to nerves and blood vessels. Conventional methods typically utilize a single rigid needle or introducer to create a pathway for the sling material. In such a manner, the path is limited by the geometry of the needle or introducer. In order to avoid risk of injury to one structure, the surgeon must often choose a pathway which risks injury to one or more less significant structures. The present inventive methods provide for the creation of a pathway that is not limited by the geometry or shape of the needle or introducer. Within the scope of the present invention, the surgeon may create a pathway having a plurality of acute and/or obtuse angles. The pathway may include changes in direction of the suture pathway, with such changes in direction occurring at angles of anywhere between 1 degree to 359 degrees. The present inventive methods allow for sling placement without the need for instrument entry into the retropubic or obturator spaces. The present inventive methods further include the option of using bodily spaces not previously utilized for sling placement and/or sling terminus locations. Such inventive methods of sling placement may negate the need for further fixation to the skin and/or fascia.

Additionally, the scope of the present inventive methods may further include the placement of synthetic material and/or natural material in the treatment of pelvic organ prolapse, fecal incontinence, and the like.

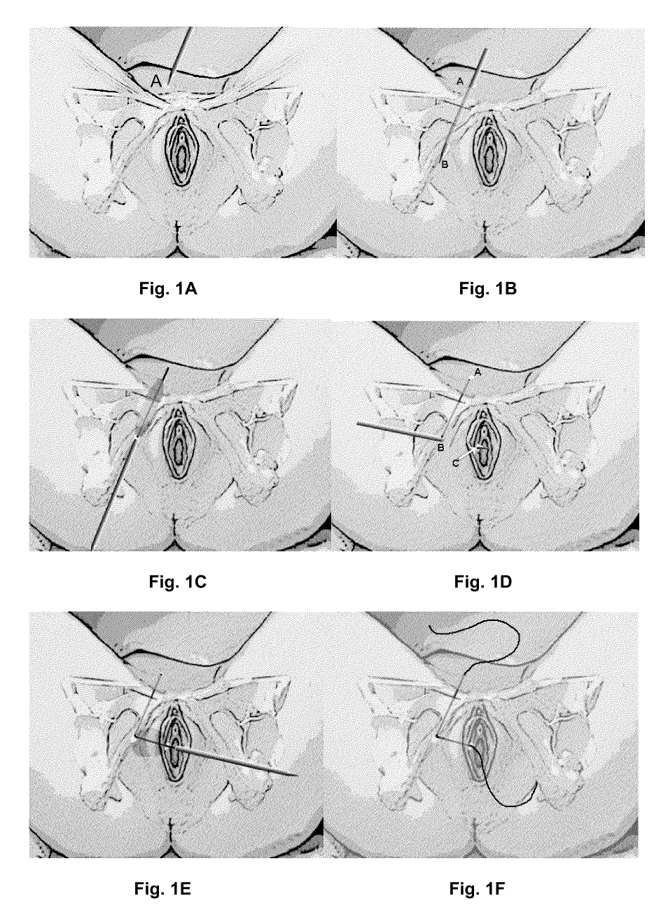

In one embodiment of a present inventive method for placing a sling or other material in a circuitous pathway through bodily tissue, a surgeon may insert a needle or introducer into a patient's body at a first insertion point A (see FIG. 1A). The needle or introducer may then be advanced through the tissue along a first trajectory, wherein the surgeon may choose a path that avoids structures of concern. The needle may then exit the bodily tissue skin at a first exit point B (see FIG. 1B-1C) pulling the sling or other material to thereafter facilitate a change in direction of both the needle and the attached sling. Alternatively, the needle or introducer may be advanced to the first exit point B without having a sling initially attached, wherein a sling is thereafter attached to the needle at the first exit point B or second exit point C and pulled back through the pathway to the initial entry point A. In a preferred embodiment, the needle or introducer may be passed beneath the skin but above the bone (e.g. pubic bone) of the patient (see FIG. 1B). The needle or introducer may then be inserted at first exit point B (see FIG. 1D) and aimed along a second trajectory that is distinct from the first trajectory between first insertion point A to first exit point B. The needle or introducer may then be advanced through the bodily tissue towards a second exit point, point C (see FIG. 1D-1E) and the sling or other material may be pulled through. Second exit point C may be the final exit point of the newly created circuitous pathway (see FIG. 1F). However, second exit point C may also serve as an intermediate exit point used for yet another change in direction (as previously shown at point B) by re-introducing the needle or introducer at the second exit point C advancing along a third trajectory to a third exit point (not shown). Such an insertion/re-introduction process may be repeated at least one time, wherein a greater number of repetitions serve to create a more tortuous sling pathway that may create a strong retention force on the sling or other material disposed there through. The inclusion of at least one additional change in direction of the sling pathway on each side of the supported tissue (e.g. urethra) serves to strengthen the sling's resistance to movement and increase the overall effectiveness of the procedure.

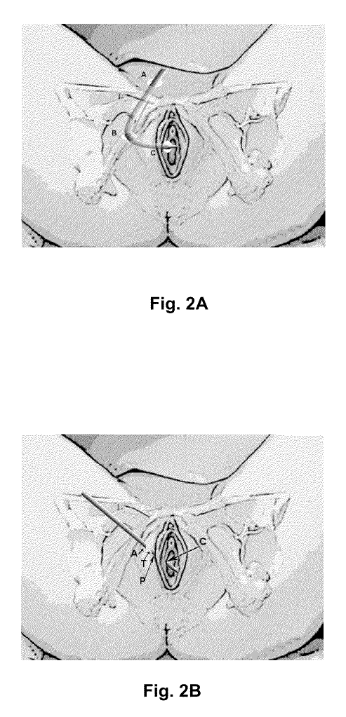

In another embodiment of the present inventive methods, the surgeon may place a malleable needle or introducer into the first insertion point A (see FIG. 2A) and advancing along a first trajectory to a first exit point B that avoids structures of concern. At any one or multiple exit points thereafter, the malleable needle or introducer may be reshaped by the surgeon (see FIG. 2A) along the path to the desired final exit point (e.g. second exit point C). The present inventive method may be done with or without the need for additional incisions (without incision, the referenced exit point need not be used for exit, but serve as points of redirecting the needle or introducer via extrinsic pressure or other means). The malleable needle or introducer may be used to push or pull the sling or other material through the circuitous pathway or the needle may carry a suture, guide, or other device, which then in turn may be used to bring the sling or other material through the pathway. The present inventive method may further be used to place and create a pathway for synthetic or natural material used to treat conditions such as pelvic organ prolapse, fecal incontinence, and the like.

The use of previously unused bodily spaces for termination points of the sling or other material, such as the supraobturator space, perineum, and the preprepubic space, may be further incorporated into the present inventive methods, including all disclosed embodiments and obvious variations thereof.

In still another embodiment, depicted in FIG. 2B, the surgeon may insert the needle or introducer at a first entry point A and advance the needle through bodily tissue to a first transient exit point T. At the first transient exit point T, the needle or introducer may undergo a rapid exit and reentry into the skin or mucosa at a second insertion point P to continue advancing to a second exit point C. The first transient exit point T and subsequent second insertion point P may comprise either the same single incision point or two distinct incision points. When two distinct incision points are used the separate incisions may comprise a rapid exit and reentry of the tip of the needle relative to the surface of the bodily tissue. Such a quick out-and-in penetration of the needle or introducer may otherwise be known as “button holing”. In the instance of separate incisions or “button holing”, the skin or mucosal bridge between the first transient exit point T and the second insertion point P may preferably be incised to allow the sling or other material pulled through the pathway to rest beneath the skin or mucosa.

The entry and exit points in all included Figures are for illustrative purposes only. Such entry and exit points may reside anywhere on the skin or mucosa of the patient, and the scope of the present invention is not limited by the examples described or depicted herein.

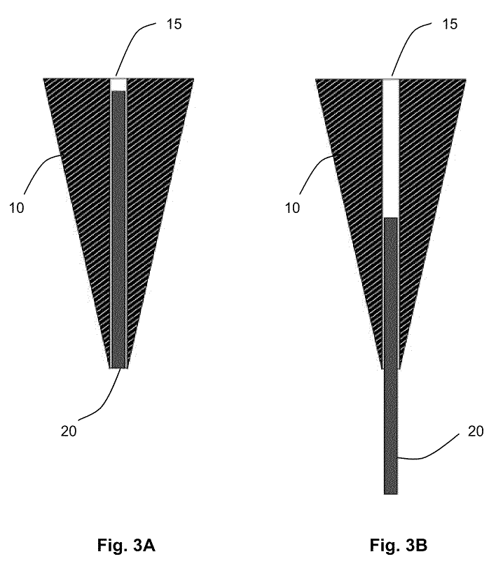

Yet another embodiment of the present inventive method allows a surgeon to utilize a tapered needle body or other device capable of creating a circuitous and tapered pathway or channel for a sling or other material. This embodiment may comprise the use of a tapered needle body or other device that may advance a thinner member to create the distal portion of the tapered needle pathway or channel for a sling or other material. The distal portion of the tapered needle pathway or channel may be disposed immediately adjacent the surface of the skin where the sling or other material may exit the body. As depicted in FIG. 3A, the tapered needle body 10 may comprise a central axial bore 15 containing an inner member 20 that may be capable of distal axial advancement. In this manner, the profile of either the tapered needle body 10 or the distally advanced inner ember 20 may serve to create the distal portion of the tapered channel or pathway for retaining a sling or other device. The distally tapered needle body 10 or inner member 20 may also serve as either a delivery and/or retrieval device for the sling or other material placed in the pathway.

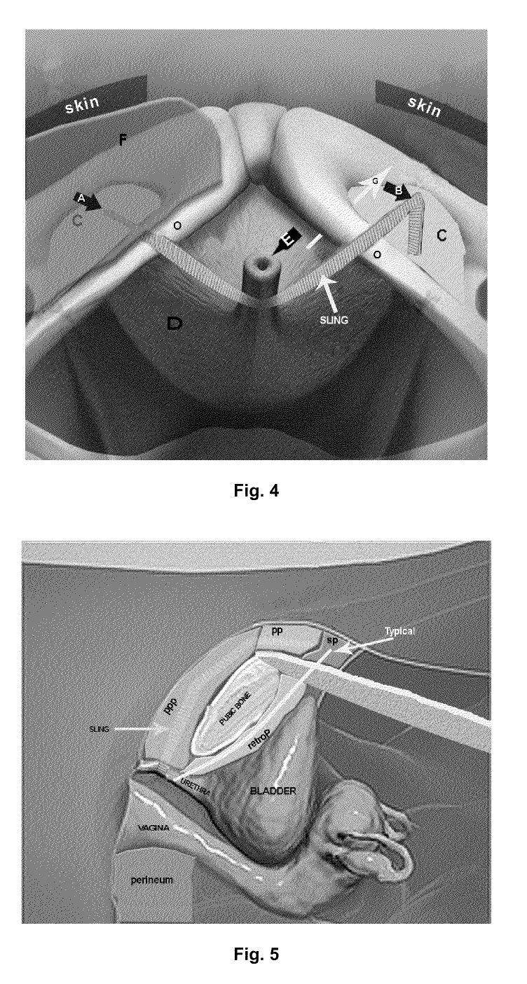

As depicted in FIG. 4 and as provided for by the tapered shape of the distal portion A of the created tapered needle pathway, the sling or other material will be squeezed, have restricted movement, and/or be further constrained in the narrower, distal portion A of the tapered needle pathway. After utilizing the disclosed distally tapered needle body 10 and/or method, the inserted sling or other material will be much less prone to post-surgical movement. In this manner, the present inventive methods and devices provide a more reliable and effective procedure for the placement of a sling or other material within the body. The present inventive method may further be used to place and create such a tapered distal pathway for synthetic or natural material used to treat conditions such as pelvic organ prolapse, fecal incontinence, and the like. The use of previously unused bodily spaces for termination points of the sling or other material, such as the supraobturator space, perineum, and the preprepubic space, and any obvious variations thereof may also be incorporated into the present invention.

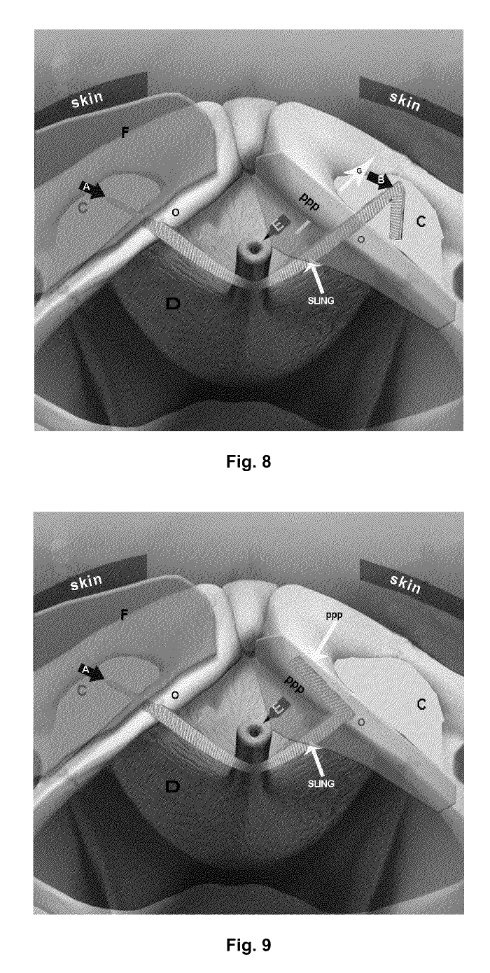

As further depicted in FIGS. 4 and 8, improved sling retention and resistance to post-surgical movement may also be enhanced via distal angulation B of the sling pathway. Such distal angulation B may serve to create a more tortuous and circuitous sling pathway within the scope of the present invention. Creation of such a circuitous pathway may have a positive effect on the success rate of a wide variety of surgical procedures requiring fixation of a sling or other material.

In still another embodiment, the surgeon may utilize an introducer comprising a delivery device of fixed shape and an extension piece made of a memory alloy or other memory material capable of creating or continuing along a path different than the path forged by the delivery device. This allows for the creation of a unique path without the need for multiple skin or mucosal incisions. A first initial trajectory is formed by the delivery device, and a second trajectory that is distinct from the first trajectory is formed via the distal advancement of the memory material extension piece. Such a procedure may be repeated along the same pathway to create two or more distinct trajectories. The combination of two or more distinct trajectories creates a circuitous pathway that is capable of providing a retentive force on the sling or other material dispose there through. As the total number of distinct pathway trajectories increases, the level of retentive force applied to the sling or other material also increases.

The needle body, inner member, delivery device, or memory shape extension may be used to push or pull the sling or other material through the created pathway or may simply carry a suture, guide, or other device that in turn is used to bring the sling into the created pathway. The present inventive methods may further be used to place and create a pathway for synthetic or natural material used to treat conditions such as pelvic organ prolapse, urinary incontinence, fecal incontinence, and the like.

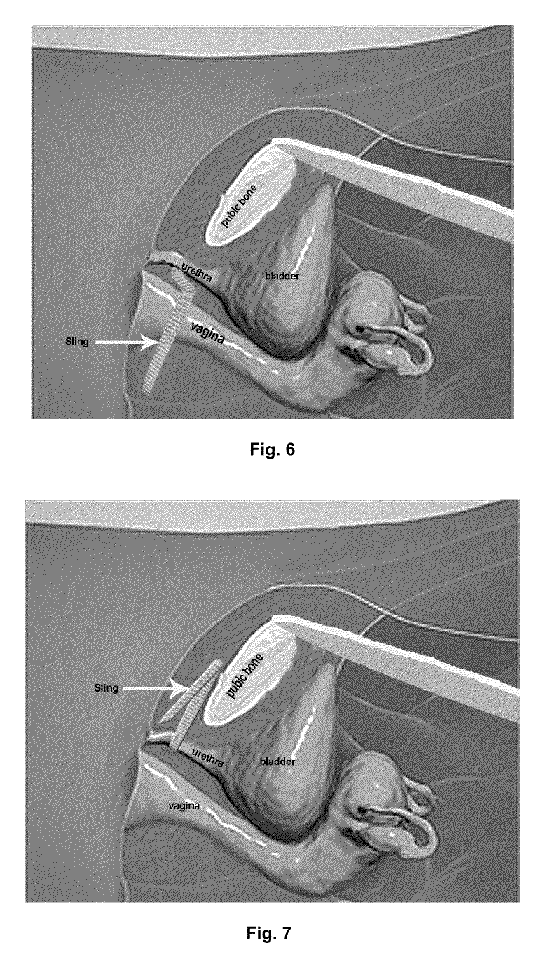

The scope of the present inventive methods further allow for the use of bodily spaces previously not considered for terminus locations of sling ends and other materials. Given the additional resistance of a sling or other placed material to movement due to the disclosed methods involving the creation of a circuitous pathway (see FIGS. 1A-1F), malleable needle (FIG. 2A), transient exit (FIG. 2B), tapered distal pathway (FIGS. 3A-3B), or introducer and extension methods, previously unused bodily regions may now be utilized due to the mootness of conventional sling fixation (e.g. suture and/or bone anchors). Such previously unusable bodily spaces may include but are not limited to the supraobturator space F (see FIGS. 4 and 8-11), the perineum (see FIG. 6), and the preprepubic space ppp (see FIGS. 7, 9, and 10).

As previously noted, certain anatomical spaces have not heretofore been utilized for conventional sling procedures and the like. These spaces may include but are not limited to the perineum, supraobturator space F, and the preprepubic space ppp. Such anatomical spaces may be difficult for a surgeon to access from the area of the urethra and/or the surgeon may be concerned that such anatomical spaces have not traditionally provided enough support or retentive strength to achieve an acceptable cure rate for the disorder being treated. The same concerns and detrimental issues have also been true for the placement of synthetic or natural material used to treat pelvic organ prolapse, fecal incontinence, and the like. Likewise, given the use of the present inventive methods and/or devices, the newly utilizable anatomical spaces may now be employed as effective terminus points for sling procedures as well as any other applicable medical procedures.

As illustrated in FIG. 4, conventional sling procedures (G arrow) within the prior art typically trace a path behind the pubic bone O and penetrate the patient's obturator muscle and/or membrane C. Thereafter, as illustrated in FIG. 5, the typical sling pathway may place the terminus of a sling or other material within the suprapubic space (sp) or retropubic space (retroP). When using the devices or methods of the present invention, sling termination may now be accomplished within the preprepubic space (ppp) as shown in FIG. 5 and within the perineum as shown in FIG. 6.

The present inventive methods allow the perineum to be utilized as an effective terminus for the ends of an inserted sling or other material. As depicted in FIG. 6, the perineum (the area between the anus and the rectum) may now be utilized as the final resting place for the sling ends or other material. The surgeon may make an incision or penetration in the skin of the perineum that serves as an exit or entrance point for a needle or introducer that enters or exits in an area reachable through a vaginal incision. Use of the perineum as the sling termination site may be further facilitated by incorporation of the inventive circuitous pathway techniques (see FIGS. 1A-1F), malleable needle (FIG. 2A), transient exit (FIG. 2B), tapered distal pathway (FIGS. 3A-3B), introducer and extension methods, and/or other methods including a single pass of a needle or introducer. Terminal sling perineum sites may also be used for placement of synthetic or natural material used to treat conditions such as pelvic organ prolapse, fecal incontinence, and the like.

The present inventive methods further allow the supraobturator space F to be utilized as an effective terminus for the ends of an inserted sling or other material. As depicted in FIGS. 4 and 8-11, the supraobturator space F (the area of soft tissue between the obturator fascia and the overlying skin) may now be utilized as the final resting of the sling ends or other material. In this method the sling terminates in the supraobturator space F but does not travel through the obturator foramen or enter the obturator muscle C. In this way, most complications incurred with conventional sling procedures (shown as arrow G in FIGS. 4 and 8) may be completely averted. In this method, the surgeon may make an incision or penetration in the skin overlying the obturator foramen that serves as an exit or entrance point for a needle or introducer that enters or exits in an area reachable through a vaginal incision or penetration that never passes through the obturator foramen. Use of the supraobturator space F as the sling termination site may be further facilitated by incorporation of the inventive circuitous pathway (see FIGS. 1A-1F), malleable needle (FIG. 2A), transient exit (FIG. 2B), tapered distal pathway (FIGS. 3A-3B), introducer and extension methods, and/or other methods including a single pass of a needle or introducer.

Additionally, FIGS. 4 and 8 depict one terminal end of the sling or other material being disposed through a circuitous distally angulated pathway B within the supraobturator space F for enhanced fixation. Additionally or alternatively, FIGS. 4 and 8-11 also depict the use of a distally tapered pathway A for the other sling termination sites within the supraobturator space F where the sling does not pass through the obturator foramen. The presently disclosed sling fixation methods, pathways, and devices may be utilized independently or in any combination thereof. Terminal sling supraobturator sites may also be used for placement of synthetic or natural material used to treat conditions such as pelvic organ prolapse, fecal incontinence, and the like.

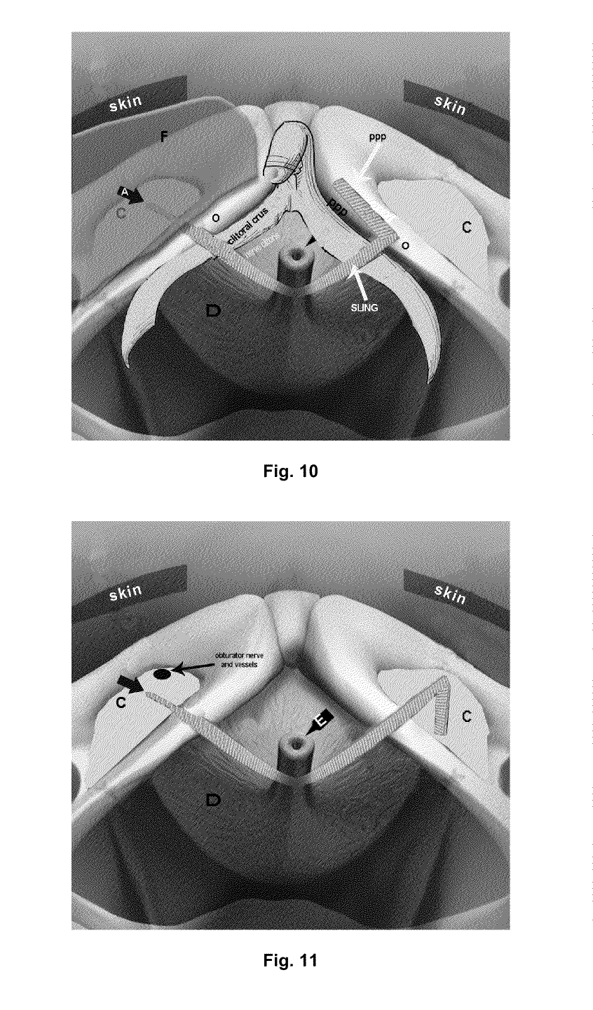

The present inventive methods yet further allow the preprepubic space (ppp) to be utilized as an effective terminus for the ends of an inserted sling or other material. As depicted in FIGS. 7, 9, and 10, the preprepubic space ppp (the subcutaneous space outlined in part by the ventral boarder of the symphysis pubis and the medial and ventral boarders of the obturator foramen) may now be utilized as the terminal location for sling ends or other material. In this way, most complications incurred with conventional sling procedures may be completely averted. Use of the preprepubic space ppp as the sling termination site may be further facilitated by incorporation of the inventive circuitous pathway (see FIGS. 1A-1F), malleable needle (FIG. 2A), transient exit (FIG. 2B), tapered distal pathway (FIGS. 3A-3B), introducer and extension methods, and/or other methods including a single pass of a needle or introducer. As illustrated in FIGS. 10-11, by selecting a sling pathway above the pubic bone O and not penetrating the obturator foramen, a surgeon may remove the risk of dorsal nerve, clitoral crus, or obturator vessel injury, respectively. The presently disclosed sling fixation methods may be used independently or in any combination thereof. Terminal sling preprepubic sites may also be used for placement of synthetic or natural material used to treat conditions such as pelvic organ prolapse, fecal incontinence, and the like.

Within the scope of the present invention, each of the disclosed methods may be performed as either a puncture or non-puncture embodiment. In a puncture embodiment, the sling ends exit through a skin incision or puncture. In a non-puncture embodiment, the sling ends do not exit through a skin incision or puncture. Each of the disclosed methods may also be performed from entry points located either outside the vagina or inside the vagina. Each of the disclosed methods and their illustrative embodiments may be easily adapted to beneficially treat urinary incontinence, fecal incontinence, vaginal prolapse, and all similar disorders known within the art.

The needles or introducers utilized in the present invention may further comprise eyelets or apertures at one or both ends of the needle for capturing a sling, suture, or other material. One or both needle ends may comprise at least one protuberance that may fit into at least one complimentary (e.g. male-to-female) receiver area in a handle. Alternatively, the needle ends may have defects which may serve as receivers for at least one complimentary protuberance disposed on the handle. Such a relationship created between the handle and needle preferably prevents rotation of the needle while it is disposed within the handle. The needle or introducer may further comprise a bulbous area or enlargement near the tip that will provide a friction fit or other such communication or engagement between the needle and a material to be push, pulled, or otherwise motivated by the needle. Each of the above features may be utilized independently or in any combination thereof on a respective needle or introducer.

While the above description contains much specificity, these should not be construed as limitations on the scope of any embodiment, but as exemplifications of the presently preferred embodiments thereof. Many other ramifications and variations are possible within the teachings of the various embodiments.

Thus the scope of the invention should be determined by the appended claims and their legal equivalents, and not by the examples given.

What is claimed is:

1. A method of placing material attached to a needle in a circuitous pathway through bodily tissue, comprising the steps of:
inserting said needle into said bodily tissue at a first insertion point;
advancing said needle in a first trajectory through said bodily tissue;
exiting said needle from said bodily tissue at a first exit point;
inserting said needle into said bodily tissue at said first exit point;
advancing said needle in a second trajectory through said bodily tissue, wherein said second trajectory is distinct from said first trajectory; and
exiting said needle from said bodily tissue at a second exit point.

2. The method of claim 1, further comprising the steps of:
inserting said needle into said bodily tissue at said second exit point;
advancing said needle in a third trajectory through said bodily tissue; wherein said third trajectory is distinct from said second trajectory; and
exiting said needle from said bodily tissue at a third exit point.

3. The method of claim 1, wherein said material is a sling.

4. The method of claim 1, wherein respective ends of said material are disposed in a location selected from the group consisting of the perineum, the supraobturator space, and the preprepubic space.

5. The method of claim 4, wherein said material does not pass through the obturator foramen.

6. The method of claim 1, wherein said needle comprises malleable material.

7. The method of claim 6, wherein said needle comprising said malleable material is reshaped prior to said inserting said needle into said bodily tissue at said first exit point facilitating creation of said circuitous pathway.

8. The method of claim 6, further comprising the step of:
reshaping said needle comprising said malleable material while said needle is disposed within said bodily tissue, wherein said advancing of said needle thereafter occurs along an altered trajectory that is distinct from said first trajectory or said second trajectory.

9. The method of claim 1, wherein said needle comprises a tapered needle body and said tapered needle body creates a tapered needle pathway for trapping said material immediately adjacent said first exit point or said second exit point, wherein the narrowest diameter of said tapered needle pathway is disposed immediately adjacent said first exit point or said second exit point.

10. The method of claim 9, wherein said tapered needle body further comprises a central axial bore having an inner member capable of axial movement disposed therein, wherein said inner member acts to deliver or retrieve said material beyond a distal tip of said tapered needle body.

11. The method of claim 1, wherein said circuitous pathway does not pass through the obturator foramen.

12. The method of claim 1, further comprising the step of:
distally advancing an extension piece from within the distal tip of said needle, wherein said extension piece comprises a memory material having a bias to conform to a resting configuration when not confined within said needle, wherein said bias creates an altered first trajectory that is distinct from said first trajectory upon said distal advancement of said extension piece from within said distal tip of said needle thereby creating said circuitous pathway without the requirement of additional incisions.

13. A method of placing material attached to a tapered needle body in a circuitous pathway through bodily tissue, comprising the steps of:
inserting said tapered needle body into said bodily tissue at a first insertion point;
advancing said tapered needle body in a first trajectory through said bodily tissue;
creating a tapered needle pathway within said bodily tissue adjacent a first exit point; and
advancing said material through said tapered needle pathway within said bodily tissue, wherein said tapered needle pathway constricts or restrains movement of said material disposed there through.

14. The method of claim 13, wherein respective ends of said material are disposed in a location selected from the group consisting of the perineum, the supraobturator space, and the preprepubic space.

15. The method of claim 14, wherein said material does not pass through the obturator foramen.

16. The method of claim 13, wherein said tapered needle body comprises malleable material allowing a user to reshape said tapered needle body as desired.

17. The method of claim 13, wherein said tapered needle body further comprises a central axial bore having an inner member capable of axial movement disposed therein, wherein said inner member acts to deliver or retrieve said material beyond a distal tip of said tapered needle body.

18. The method of claim 13, wherein said circuitous pathway does not pass through the obturator foramen.

19. The method of claim 13, further comprising the step of:
distally advancing an extension piece from within the distal tip of said tapered needle body, wherein said extension piece comprises a memory material having a bias to conform to a resting configuration when not confined within said tapered needle body, wherein said bias creates an altered first trajectory that is distinct from said first trajectory upon said distal advancement of said extension piece from within said distal tip of said tapered needle body thereby creating said circuitous pathway without the requirement of additional incisions.

20. A method of placing material attached to a needle in a circuitous pathway through bodily tissue, comprising the steps of:
inserting said needle into said bodily tissue at a first insertion point;
advancing said needle into said bodily tissue;
exiting said needle from said bodily tissue at a first transient exit point;
inserting said needle into said bodily tissue at a second insertion point;
advancing said needle into said bodily tissue; and
exiting said needle from said bodily tissue at a second exit point.

21. The method of claim 20, wherein said first transient exit point and said second insertion point comprise a single incision point.

22. The method of claim 20, wherein said first transient exit point and said second insertion point comprise two distinct incision points, wherein said two distinct incision points are created via a rapid exit and reentry of the tip of said needle with respect to said bodily tissue.

23. The method of claim 22, wherein the skin or mucosal bridge between said first transient exit point and said second insertion point is incised allowing said material pulled through said circuitous pathway to rest beneath said skin or said mucosal bridge.

24. The method of claim 20, wherein said circuitous pathway does not pass through the obturator foramen.

25. The method of claim 20, wherein respective ends of said material are disposed in a location selected from the group consisting of the perineum, the supraobturator space, and the preprepubic space.

26. The method of claim 25, wherein said material does not pass through the obturator foramen.

27. The method of claim 20, further comprising the step of:
distally advancing an extension piece from within the distal tip of said needle, wherein said extension piece comprises a memory material having a bias to conform to a resting configuration when not confined within said needle, wherein said bias creates an altered first trajectory that is distinct from an initial first trajectory upon said distal advancement of said extension piece from within said distal tip of said needle thereby creating said circuitous pathway without the requirement of additional incisions.
